(19) United States
(12) Patent Application Publication
(10) Pub. No.: US 2010/0272781 A1
(43) Pub. Date: Oct. 28, 2010

(54) Sugar Free Chewable Tablets

(76) Inventors: Veerappan Subramanian, Mendham, NJ (US); Muthusamy Shanmugan, Monmouth Junction, NJ (US); Yuh-Herng Eric Lin, East Brunswick, NJ (US); Kanni Paranjothy, Mendham, NJ (US); Kranthikumar Gorlamari, North Plainfield, NJ (US)

Correspondence Address: Barbara V. Maurer, 5 Haven Hill Rise, Flemington, NJ, 08822, US

(21) Appl. No.: 12/749,482

(22) Filed: Mar. 29, 2010

Related U.S. Application Data
(60) Provisional application No. 61/212,050, filed on Apr. 6, 2009.

(51) Int. Cl. A61K 9/20; A61K 9/28; A61K 31/7016; A61K 33/08; A61K 33/10; A61K 31/4164; A61K 31/341; A61K 31/426; A61P 1/04; A61P 7/00

(52) U.S. Cl. 424/441; 514/25; 424/692; 424/687; 514/400; 514/471; 514/370; 514/365

(57) ABSTRACT

This invention relates to chewable tablets containing medicants such as gastrointestinal drugs. More particularly, the invention relates to chewable tablets of medicaments that are sugar free and have reduced calories. Accordingly, the tablets of the invention are suitable for both diabetic and non-diabetic patients.

This application claims priority of U.S. provisional application Ser. No. 61/212,050, filed Apr. 6, 2009, incorporated herein by reference in its entirety.

This invention relates to chewable tablets containing medicants such as gastrointestinal drugs. More particularly, the invention relates to chewable tablets of medicaments that are sugar free and have reduced calories. Accordingly, the tablets of the invention are suitable for both diabetic and non-diabetic patients.

Chewable tablets are taken slowly by chewing or sucking in the mouth, and enable the pharmaceutical content therein to be orally administered without water. Considering the patient's convenience and acceptance, chewable tablets would be preferred over the other dosage forms. Suspension type of products, although convenient for consumption at home, may pose problems in the case of transport outside the home.

More particularly tablets containing one gram or more of the active ingredient are large and may be difficult for patients to swallow. When the tablets are smaller and have a correspondingly reduced amount of medicament, the number of tablets would have to be greatly increased in order to achieve the required daily dose.

Thus there remains a need for new chewable tablets of medicaments that are easy for a patient to use and have as few calories as possible.

It has now been found that chewable tablets having Isomalt as a constituent meet the objectives of the invention.

Further aspects of the invention are set forth in the accompanying specification and examples.

This invention relates to a sugar-free chewable medicament tablets.

The sweeteners used in the invention comprise Isomalt and other non-calorie sweeteners in combination and are of unique nature. Isomalt is low calorie natural sweetener and an excellent sugar substitute. It provides palatable taste, texture and half the calories of the sugar. Thus the chewable sucralfate tablets of the invention give a pleasant mouth feel and bring relief to the ailing patients without adding to the caloric intake of the patient. Accordingly, the sugar free chewable sucralfate tablet formulation of the invention is a benefit to both diabetic and non-diabetic patients.

Other non-caloric sweeteners that can be used in combination with Isomalt include aspartame, acesulfame K, saccharin, and the like.

Medicaments that can be used in the formulation of the invention include those that are typically given in larger doses and are generally stable to water or other other substances in the human body until the medicament reaches the area in the body where it is absorbed. More particularly medicaments of the invention include drugs that are associated with the treatment of duodenal ulcer, gastroesophageal reflux disease, stomach ulcer, stress ulcer prophylaxis, duodenal ulcer prophylaxis and hyperphosphatejia of renal failure. Examples of these drugs include minerals and electrolytes such as magnesium oxide, antacids such as calcium carbonate, H2 antagonists such as cimetidine, rantidine, famotidine or nizatidine and miscellaneous GI agents such as sucralfate. It is possible, where appropriate, to add natural or synthetic flavorings, fragrances, auxiliaries and excipients such as, for example, cherry flavoring or magnesium stearate.

It is also possible to add other active ingredients to the sucralfate chewable tablets according to the invention. Suitable ingredients, which are known to be combinable with sucralfate, are, for example, antacids, spasmolytics, antiflatulents, beta-2 receptor blockers, non-steroidal antirheumatics and generally drugs which inhibit acid secretion.

The range of medical uses of the chewable tablet according to the invention is entirely analogous to the known dosage forms of medicament which is well known in the art.

The pharmaceutical compositions according to the invention can be produced by first mixing the medicament with a sugar alcohol such as mannitol or sorbitol, the sugar substitute such as aspartame or Isomalt, sodium starch glycolate and pregelatinized starch to form a dry mixture. Then one or more granulating agents, coloring agent and an excipient such as Povidone K 30 or coprovidone, which have been dissolved in water are added and the mixture is granulated and dried. The dried granules are then milled and additional super disintegrants and flavorings may be added. A lubricant such as magnesium stearate is added and mixed and the mixture is then compressed into the tablets of the invention.

As an example of one embodiment of the invention, the chewable sucralfate tablet of the invention contains about 1 gram of sucralfate. It may be difficult sometimes for some patients to swallow the whole tablet as the size is bigger. Chewable sucralfate tablets are convenient, can be kept in the buccal cavity, chewed and be taken with the pleasant mouth feel.

Sucralfate, used for producing the sucralfate chewable tablet according to the invention, is a conventional, commercially available active ingredient. Preferably sucralfate is employed in finely ground form with a particle size of preferably below about 50 μm. About 750 to 1,250 mg, preferably, 1,000 mg of sucralfate is employed per tablet.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### Example 1

| Item # | Ingredient | Mg/tablet |
|---|---|---|
| 1 | Sucralfate USP | 1000.00 |
| 2 | Mannitol | 600.95 |
| 3 | Aspartame | 18.00 |
| 4 | Sodium Starch Glycolate | 16.00 |
| 5 | Pregelatinized Starch | 63.00 |
| 6 | Povidone K 30 | 72.00 |
| 7 | FD&C Red # 40 | 0.05 |
| 8 | Mixed berry flavor | 12.00 |
| 9 | Magnesium Stearate | 18.00 |

Process:
1. Items 1, 2, 3, 4 and 5 are mixed well in a granulator.
2. Items 6 and 7 are dissolved in water and used as a granulating agent.
3. The solution from Step 2 is added to the mixture in Step 1 and granulated well
4. The wet mass is dried until the moisture in the mixture is about the same as the pre-blend sample.
5. The dried granules are milled and item 8 is added and mixed in a blender for 10 minutes.
6. Item 9 is added the mixture is lubricated for 5 minutes.
7. The granules are compressed into the tablets of the invention.

### Example 2

| Item # | Ingredient | Mg/tablet |
|---|---|---|
| 1 | Sucralfate USP | 1000.00 |
| 2 | Isomalt | 100.00 |
| 3 | Mannitol | 500.95 |
| 4 | Aspartame | 18.00 |
| 5 | Sodium Starch Glycolate | 16.00 |
| 6 | Pregelatinized Starch | 63.00 |
| 7 | Povidone K 30 | 72.00 |
| 8 | FD&C Red # 40 | 0.05 |
| 9 | Mixed berry flavor | 12.00 |
| 10 | Magnesium Stearate | 18.00 |

Process:
1. Items 1, 2, 3, 4, 5 and 6 are mixed well in a granulator.
2. Items 7 and 8 are dissolved in water and used as a granulating agent.
3. The solution from Step 2 is added to the mixture in Step 1 and granulated well
4. The wet mass is dried until the moisture in the mixture is about the same as the pre-blend sample.
5. The dried granules are milled and item 9 is added and mixed in a blender for 10 minutes.
6. Item 10 is added the mixture is lubricated for 5 minutes.
7. The granules are compressed into the tablets of the invention.

The preceding examples can be repeated with similar success by substituting the generically or specifically described reactants and/or operating conditions of this invention for those used in the preceding examples.

From the foregoing description, one skilled in the art can easily ascertain the essential characteristics of this invention, and without departing from the spirit and scope thereof, can make various changes and modifications of the invention to adapt it to various usages and conditions.

1. A pharmaceutical composition comprising a medicament and at least one pharmaceutically acceptable sugar free sweetener, in the form of a chewable tablet or a chewable coated tablet.

2. The pharmaceutical composition of claim 1 wherein the sugar free sweetener is a sugar alcohol.

3. The pharmaceutical composition of claim 2 wherein the sugar alcohol is Isomalt.

4. The pharmaceutical composition of claim 1 wherein the medicament is associated with the treatment of duodenal ulcer, gastroesophageal reflux disease, stomach ulcer, stress ulcer prophylaxis, duodenal ulcer prophylaxis or hyperphosphatejia of renal failure.

5. The pharmaceutical composition of claim 4 wherein the medicament is mineral, electrolyte, antacid, H2 antagonist, or GI agent.

6. The pharmaceutical composition of claim 5 wherein the medicament is magnesium oxide, calcium carbonate, sucralfate, cimetidine, rantidine, famotidine or nizatidine.

7. The pharmaceutical composition of claim 3 wherein the medicament is sucralfate.
